(19) United States
(12) Patent Application Publication
(10) Pub. No.: US 2004/0266857 A1
(43) Pub. Date: Dec. 30, 2004

(54) Novel lapachone compounds and methods of use thereof

(76) Inventors: Zhiwei Jiang, Grafton, MA (US); Dasharatha Reddy, Acton, MA (US); Samuel K. Ackerman, Weston, MA (US); June Salvesen, Marlborough, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/810,260

(22) Filed: Mar. 26, 2004

Related U.S. Application Data
(63) Continuation of application No. PCT/US03/37219, filed on Nov. 18, 2003.
(60) Provisional application No. 60/427,283, filed on Nov. 18, 2002.

(51) Int. Cl. A61K 31/382; C07D 335/10

(52) U.S. Cl. 514/437; 549/26

(57) ABSTRACT

The invention provides lapachone analogs and derivatives as well as methods of use thereof. These compounds can be used in pharmaceutical compositions for the treatment or prevention of cell proliferation disorders. These compounds can also be used in the treatment or prevention of psoriasis or cancer or precancerous conditions.

### BACKGROUND OF THE INVENTION

With more than 563,000 deaths in the United States annually, cancer is the second leading cause of death behind heart disease (UBS Warburg “Disease Dynamics: The Cancer Market,” Nov. 8, 2000). Surgery and radiotherapy may be curative if the disease is found early, but current drug therapies for metastatic disease are mostly palliative and seldom offer a long-term cure. Even with the new chemotherapies entering the market, improvement in patient survival is measured in months rather than in years, and the need continues for new drugs effective both in combination with existing agents as first line therapy and as second and third line therapies in treatment of resistant tumors.

In the past, the most successful drug treatment regimens have combined two or more agents, each of which has a different mechanism of action and each of which has antitumor activity when used individually. Even though their mechanisms of action differ, most of the agents currently used for chemotherapy of cancer, including alkylating agents, platinum analogs, anthracyclines and the camptothecin family of topoisomerase inhibitors, have the property of severely damaging DNA in common, hence their designation as “DNA-damaging agents.” Radiotherapy works similarly. Most DNA-damaging agents as well as the microtubule-targeting agents (e.g., paclitaxel) cause the arrest of cells at the G₂/M transition phase of the cell cycle, a major cell cycle checkpoint where cells make a commitment to repair DNA or to undergo apoptosis if DNA damage is irreparable. Recently, interest has grown in identifying new therapeutic agents to further exploit cell checkpoint functions.

β-lapachone (3,4-dihydro-2,2-dimethyl-2H-naphtho[1,2-b]pyran-5,6-dione), a quinone, is derived from lapachol (a naphthoquinone) which can be isolated from the lapacho tree (Tabebuia avellanedae), a member of the catalpa family (Bignoniaceae). Lapachol and β-lapachone (with numbering) have the following chemical structures:

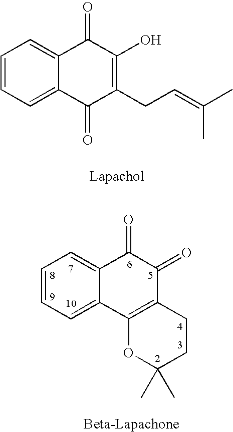

β-lapachone, as well as its intermediates, derivatives and analogs thereof, are described in Li, C. J. et al., (1993) J. Biol. Chem., 268(30): 22463-22468. As a single agent, β-lapachone has demonstrated significant antineoplastic activity against human cancer cell lines at concentrations typically in the range of 1-10 μM (IC₅₀). Cytotoxicity has been demonstrated in transformed cell lines derived from patients with promyelocytic leukemia (Planchon et al., (1996) Cancer Res., 55: 3706-3711), prostate (Li, C. J., et al., (1995) Cancer Res., 55: 3712-3715), malignant glioma (Weller, M. et al., (1997) Int. J. Cancer, 73: 707-714), hepatoma (Lai, C. C., et al., (1998) Histol Histopathol, 13: 89-97), colon (Huang, L., et al., (1999) Mol Med, 5,: 711-720), breast (Wuertzberger, S. M., et al., (1998) Cancer Res., 58: 1876), ovarian (Li, C. J. et al., (1999) Proc. Natl. Acad. Sci. USA, 96(23): 13369-13374), pancreatic (Li, Y., et al., (2000) Mol Med, 6: 1008-1015; Li, Y., (1999) Mol Med, 5: 232-239), and multiple myeloma cell lines, including drug-resistant lines (Li, Y., (2000) Mol Med, 6: 1008-1015). No cytotoxic effects were observed on normal fresh or proliferating human PBMC (Li, Y., (2000) Mol Med, 6: 1008-1015).

β-lapachone appears to work by inducing unscheduled expression of checkpoint molecules, e.g. E2F, independent of DNA damage and cell cycle stages. Several studies have shown that β-lapachone activates checkpoints and induces apoptosis in cancer cells from a variety of tissues without affecting normal cells from these tissues (U.S. Patent Application Publication No. 2002/0169135, incorporated by reference herein). In normal cells with their intact regulatory mechanisms, such an imposed expression of a checkpoint molecule results in a transient expression pattern and causes little consequence. In contrast, cancer and pre-cancer cells have defective mechanisms, which result in unchecked and persistent expression of unscheduled checkpoint molecules, e.g. E2F, leading to selective cell death in cancer and pre-cancer cells.

In addition to β-lapachone, a number of β-lapachone analogs having antiproliferative properties have been disclosed in the art, such as those described in PCT International Application PCT/US93/07878 (WO94/04145), which is incorporated by reference herein, and U.S. Pat. No. 6,245,807, incorporated by reference herein, in which a variety of substituents may be attached at positions 3- and 4- on the β-lapachone compound. PCT International Application PCT/US00/10169 (WO 00/61142), incorporated by reference herein, discloses β-lapachone, which may have a variety of substituents at the 3-position as well as in place of the methyl groups attached at the 2-position. U.S. Pat. Nos. 5,763,625, 5,824,700, and 5,969,163, each of which is incorporated by reference herein, disclose analogs and derivatives with a variety of substituents at the 2-, 3- and 4-positions. Furthermore, a number of journals report β-lapachone analogs and derivatives with substituents at one or more of the following positions: 2-, 3-, 8- and/or 9-positions, (See, Sabba et al., (1984) J Med Chem 27:990-994 (substituents at the 2-, 8- and 9-positions); (Portela and Stoppani, (1996) Biochem Pharm 51:275-283 (substituents at the 2- and 9-positions); Goncalves et al., (1998) Molecular and Biochemical Parasitology 1:167-176 (substituents at the 2- and 3-positions)).

Moreover, structures having sulfur-containing hetero-rings in the “α” and “β” positions of lapachone have been reported (Kurokawa S, (1970) Bulletin of The Chemical Society of Japan 43:1454-1459; Tapia, R A et al., (2000) Heterocycles 53(3):585-598; Tapia, R A et al., (1997) Tetrahedron Letters 38(1):153-154; Chuang, C P et al., (1996) Heterocycles 40(10):2215-2221; Suginome H et al., (1993) Journal of the Chemical Society, Chemical Communications 9:807-809; Tonholo J et al., (1988) Journal of the Brazilian Chemical Society 9(2):163-169; and Krapcho A P et al., (1990) Journal of Medicinal Chemistry 33(9):2651-2655); however, in the majority of these compounds, the hetero-ring is unreduced, and the methods of synthesis are quite different from those of the present invention. Those known compounds having reduced sulfur-containing hetero-rings in the “α” position also report quite different methods of synthesis from the present invention, i.e. the formation of 2,3-dihydronaphtho[2,3-b]thiophene-4,9-diones by photoaddition of 2-mercapto-1,4-naphthoquinones with alkenes (Suginome et al.) or production of naphtho[2,3-b]thiopyranoquinone by an Alder-Rickert reaction of benzothiopyranoquinone with 1,3-diene (Tapia et al. 2000). These findings encourage the design and synthesis of new lapachone derivatives and analogs and their evaluation for antiproliferative activity in a variety of biological systems.

### SUMMARY OF THE INVENTION

The present invention provides the compounds of Formula I:

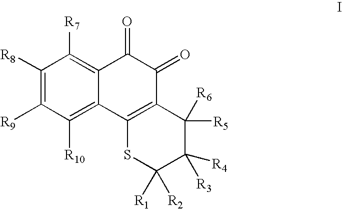

or pharmaceutically acceptable salts thereof, or a regioisomeric mixture thereof, wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R1 or R2 and one of R3 or R4; or one of R3 or R4 and one of R5 or R6 form a fused ring, wherein the ring has 4-8 ring members; R7-R10 are each, independently, hydrogen, hydroxyl, halogen, substituted or unsubstituted alkyl, substituted or unsubstituted alkoxy, nitro, cyano or amide; and n is an integer from 0 to 10.

In a preferred embodiment, R1 and R2 are alkyl, R3-R6 are, independently, H, OH, halogen, alkyl, alkoxy, substituted or unsubstituted acyl, substituted alkenyl or substituted alkyl carbonyl, and R7-R10 are hydrogen. In another preferred embodiment, R1 and R2 are each methyl and R3-R10 are each hydrogen. In another preferred embodiment, R1-R4 are each hydrogen, R5 and R6 are each methyl and R7-R10 are each hydrogen.

The present invention also provides the compounds of Formula II:

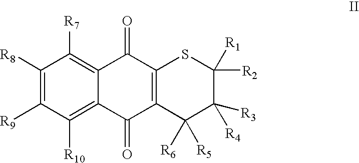

or pharmaceutically acceptable salts thereof, or a regioisomeric mixture thereof, wherein

R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R7-R10 are each, independently, hydrogen, hydroxyl, halogen, substituted or unsubstituted alkyl, substituted or unsubstituted alkoxy, nitro, cyano or amide; and n is an integer from 0 to 10,

wherein when R1 and R2 are both methyl, R3 and R4 are both H, and one of R5 and R6 is OH and the other H, R7 is not methyl or methoxy and R10 is not methyl.

In a preferred embodiment, R1 and R2 are alkyl, R3-R6 are independently H, OH, halogen, alkyl, alkoxy, substituted and unsubstituted acyl, substituted alkenyl or substituted alkyl carbonyl, and R7-R10 are hydrogen. In another preferred embodiment, R1 and R2 are each methyl and R3-R10 are each hydrogen.

The present invention also provides the compounds of Formula III:

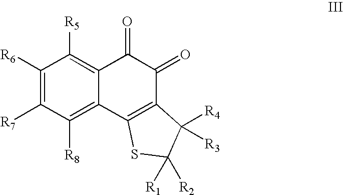

or pharmaceutically acceptable salts thereof, or a regioisomeric mixture thereof, wherein

R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R5-R8 are each, independently, hydrogen, hydroxyl, halogen, substituted or unsubstituted alkyl, substituted or unsubstituted alkoxy, nitro, cyano or amide; and n is an integer from 0 to 10.

In a preferred embodiment, one of R1 and R2 is H and the other is alkyl, R3 and R4 are independently H, OH, halogen, alkyl, alkoxy, substituted or unsubstituted alkenyl or substituted or unsubstituted alkyl carbonyl, and R5-R8 are each hydrogen. In another preferred embodiment, one of R1 and R2 is H and the other is alkyl, R3 and R4 are each methyl, and R5-R8 are each hydrogen.

The present invention also provides the compounds of Formula IV:

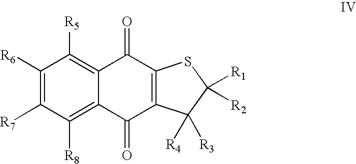

or pharmaceutically acceptable salts thereof, or a regioisomeric mixture thereof, wherein

R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R5-R8 are each, independently, hydrogen, hydroxyl, halogen, substituted or unsubstituted alkyl, substituted or unsubstituted alkoxy, nitro, cyano or amide; and n is an integer from 0 to 10,

wherein: when R1 and R2 are both methyl, one of R3 and R4 is not methyl and the other H, and each of R5, R6, R7 and R8 is not H; when R1 and R2 are both methyl, at least one of R3, R4, R5, R6, R7 and R8 is not H; when R1 and R2 are both phenyl, at least one of R3, R4, R5, R6, R7 and R8 is not H; when one of R1 and R2 is phenyl and the other is H, at least one of R3, R4, R5, R6, R7 and R8 is not H; when one of R1 and R2 is phenyl and the other is methyl, at least one of R3, R4, R5, R6, R7 and R8 is not H; when one of R1 and R2 is methyl and the other is H and R5, R6, R7 and R8 are H, at least one of R3 and R4 is not OH and the other H; when one of R3 and R4 are carboethoxy the other is H; and R1, R2, R3, R4, R5, R6, R7 and R8 are not each H.

In a preferred embodiment, both R1 and R2 are substituted or unsubstituted alkyl, R3 and R4 are independently H, OH, halogen, alkyl, alkoxy, substituted or unsubstituted alkenyl or substituted or unsubstituted alkyl carbonyl, and R5-R8 are each hydrogen. In another preferred embodiment, one of R1 and R2 is H and the other is methyl, R3 and R4 are each methyl and R5-R8 are each hydrogen.

The present invention also provides the compounds of Formula V:

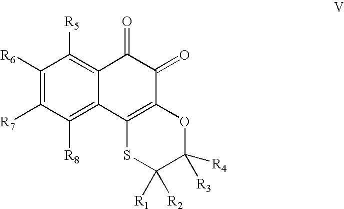

or pharmaceutically acceptable salts thereof, or a regioisomeric mixture thereof, wherein

R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R1 or R2 and one of R3 or R4 form a fused ring, wherein the ring has 4-8 ring members; R5-R8 are each, independently, hydrogen, hydroxyl, halogen, substituted or unsubstituted alkyl, substituted or unsubstituted alkoxy, nitro, cyano or amide; and n is an integer from 1 to 10,

wherein R1, R2, R3, R4, R5, R6, R7 and R8 are not each H.

In a preferred embodiment, R1 and R2 are alkyl, R3-R4 are, independently, H, OH, halogen, alkyl, alkoxy, substituted or unsubstituted acyl, substituted alkenyl or substituted alkyl carbonyl, and R7-R10 are each hydrogen. In another preferred embodiment, R1 and R2 are each hydrogen, one of R3 and R4 is methyl and the other is hydrogen and R5-R8 are each hydrogen. In another preferred embodiment, one of R1 and R2 is methyl and the other is hydrogen, one of R3 and R4 is methyl and the other is hydrogen and R5-R8 are each hydrogen. In another preferred embodiment, one of R1 and R2 is methyl and the other is hydrogen, one of R3 and R4 is hydroxymethyl and the other is hydrogen and R5-R8 are each hydrogen. In another preferred embodiment, one of R1 and R2 is methyl and the other is hydrogen, R3 and R4 are each methyl and R5-R8 are each hydrogen.

The present invention also provides pharmaceutical compositions comprising a compound of Formula I, II, III, IV or V in combination with a pharmaceutically acceptable carrier. Preferably, the compound of Formula I, II, III, IV or V is in a therapeutically effective amount.

The present invention also provides a method of treating or preventing cell proliferative disorders comprising administering to a mammal in need thereof a therapeutically effective amount of a compound of Formula I, II, III, IV or V. Preferably, administration a compound of Formula I, II, III, IV or V induces sustained elevation of E2F levels in abnormally proliferating cells without affecting E2F levels in normal cells.

The present invention also provides a method of treating cancer or precancerous conditions or preventing cancer comprising administering to a mammal in need thereof a therapeutically effective amount of a compound of Formula I, II, III, IV or V. Preferably, administration induces sustained elevation of E2F levels in cancer cells without affecting E2F levels in normal cells

The present invention also provides a method of treating or preventing psoriasis comprising administering to a mammal in need thereof a therapeutically effective amount of a compound of Formula I, II, III, IV or V.

The present invention also provides methods for the synthesis of compounds of Formula I, II, III, IV or V.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 illustrates lapachone analog and derivative compounds in accordance with the present invention.

### DETAILED DESCRIPTION OF THE INVENTION

The present invention provides novel tricyclic dihydrothiopyran and dihydrothiophene naphthoquinone derivatives, a synthetic method for making the derivatives, and the use of the derivatives to inhibit neoplastic cell proliferation. The naphthoquinone derivatives of the present invention are related to the compounds known by their trivial names as β-lapachone (3,4-dihydro-2,2-dimethyl-2H-naphtho(1,2-b)pyran-5,6-dione), α-lapachone (3,4-dihydro-2,2-dimethyl-2H-naphtho[2,3-b]pyran-5,10-dione), and dunnione (2,3,3-trimethyl-2,3,4,5-tetrahydro-naphtho(2,3-b)dihydrofuran-6,7-dione). α-lapachone and dunnione have the following chemical structures:

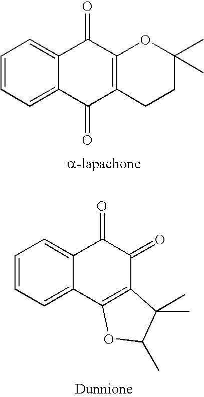

The structure of β-lapachone is described above.

The special features of the analogs of the present invention are their dihydrothiophene and dihydrothiopyran hetero-rings. In particular, there are no known naphthoquinone derivatives that have dihydrothiopyran or dihydrothiophene hetero-rings in the “β” position, i.e. analogous to β-lapachone or dunnione, respectively.

In one embodiment, the present invention provides the compounds of Formula I:

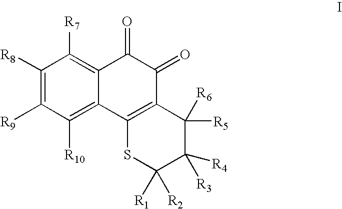

or pharmaceutically acceptable salts thereof or a regioisomeric mixture thereof, wherein

R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R1 or R2 and one of R3 or R4; or one of R3 or R4 and one of R5 or R6 form a fused ring, wherein the ring has 4-8 ring members (For Example, R1 and R3, taken together, form a 4-8 membered carbocycle or heterocycle);

R7-R10 are each, independently, hydrogen, hydroxyl, halogen, substituted or unsubstituted alkyl, substituted or unsubstituted alkoxy, nitro, cyano or amide; and

n is an integer from 0 to 10.

Preferred compounds of Formula I are those in which R1 and R2 are alkyl, R3-R6 are independently H, OH, halogen, alkyl, alkoxy, substituted and unsubstituted acyl, substituted alkenyl or substituted alkyl carbonyl, and R7-R10 are hydrogen. Preferably, R1 and R2 are each methyl and R3-R10 are each hydrogen. In another preferred embodiment, R1-R4 are each hydrogen, R5 and R6 are each methyl and R7-R10 are each hydrogen. In another preferred embodiment, R1-R4 are hydrogen, one of R5 and R6 is aryl, wherein the preferred aryl is phenyl, and the other is hydrogen, and each of R7-R10 is hydrogen.

In another embodiment, the present invention provides the compounds of Formula II:

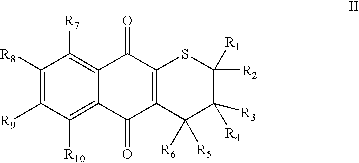

or pharmaceutically acceptable salts thereof or a regioisomeric mixture thereof, wherein

R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R7-R10 are each, independently, hydrogen, hydroxyl, halogen, substituted or unsubstituted alkyl, substituted or unsubstituted alkoxy, nitro, cyano or amide; and

n is an integer from 0 to 10,

wherein when R1 and R2 are both methyl, R3 and R4 are both H, and one of R5 and R6 is OH and the other H, R7 is not methyl or methoxy and R10 is not methyl.

Preferred compounds of Formula II are those in which R1 and R2 are alkyl, R3-R6 are independently H, OH, halogen, alkyl, alkoxyl, substituted and unsubstituted acyl, substituted alkenyl or substituted alkyl carbonyl, and R7-R10 are each hydrogen. Preferably, R1 and R2 are each methyl and R3-R10 are each hydrogen.

In another embodiment, the present invention provides the compounds of Formula III:

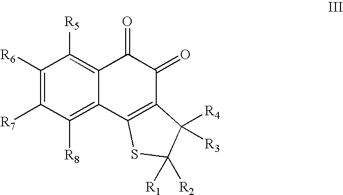

or pharmaceutically acceptable salts thereof or a regioisomeric mixture thereof, wherein

R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R5-R8 are each, independently, hydrogen, hydroxyl, halogen, substituted or unsubstituted alkyl, substituted or unsubstituted alkoxy, nitro, cyano or amide; and

n is an integer from 0 to 10.

Preferred compounds of Formula III are those in which one of R1 and R2 is H and the other is alkyl, R3 and R4 are independently H, OH, halogen, alkyl, alkoxy, substituted or unsubstituted alkenyl or substituted or unsubstituted alkyl carbonyl, and R5-R8 are each hydrogen. Preferably, one of R1 and R2 are H and the other is alkyl, R3 and R4 are each methyl and R5-R8 are each hydrogen. In another preferred embodiment, one of R1 and R2 is methyl and the other is hydrogen, R3 and R4 are each aryl, wherein the preferred aryl is phenyl and R5-R8 are each hydrogen.

In another embodiment, the present invention provides the compounds of Formula IV:

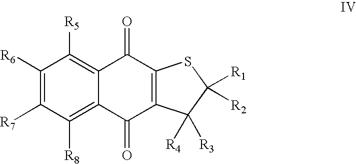

or pharmaceutically acceptable salts thereof, or a regioisomeric mixture thereof, wherein

R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R5-R8 are each, independently, hydrogen, hydroxyl, halogen, substituted or unsubstituted alkyl, substituted or unsubstituted alkoxy, nitro, cyano or amide; and

n is an integer from 0 to 10,

wherein:

when R1 and R2 are both methyl, one of R3 and R4 is not methyl and the other H (taken together, R3 and R4 are not methyl and H, in either arrangement), and each of R5, R6, R7 and R8 is not H;

when R1 and R2 are both methyl, at least one of R3, R4, R5, R6, R7 and R8 is not H;

when R1 and R2 are both phenyl, at least one of R3, R4, R5, R6, R7 and R8 is not H;

when one of R1 and R2 is phenyl and the other is H, at least one of R3, R4, R5, R6, R7 and R8 is not H;

when one of R1 and R2 is phenyl and the other is methyl, at least one of R3, R4, R5, R6, R7 and R8 is not H;

when one of R1 and R2 is methyl and the other is H and R5, R6, R7 and R8 are H, at least one of R3 and R4 is not OH and the other H (taken together, R3 and R4 are not OH and H, in either arrangement);

when one of R3 and R4 are carboethoxy the other is H; and

R1, R2, R3, R4, R5, R6, R7 and R8 are not each H.

Preferred compounds of Formula IV are those in which both R1 and R2 are substituted or unsubstituted alkyl, R3 and R4 are independently H, OH, halogen, alkyl, alkoxy, substituted or unsubstituted alkenyl or substituted or unsubstituted alkyl carbonyl, and R5-R8 are each hydrogen. Preferably, one of R1 and R2 is H and the other is alkyl, R3 and R4 are each methyl and R5-R8 are each hydrogen.

In another embodiment, the present invention provides the compounds of Formula V:

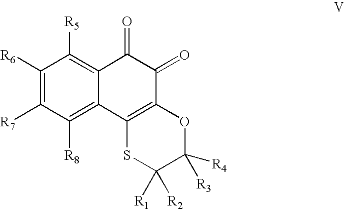

or pharmaceutically acceptable salts thereof or a regioisomeric mixture thereof, wherein

R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R1 or R2 and one of R3 or R4 form a fused ring, wherein the ring has 4-8 ring members;

R5-R8 are each, independently, hydrogen, hydroxyl, halogen, substituted or unsubstituted alkyl, substituted or unsubstituted alkoxy, nitro, cyano or amide; and

n is an integer from 1 to 10,

wherein R1, R2, R3, R4, R5, R6, R7 and R8 are not each H.

Preferred compounds of Formula V are those in which R1 and R2 are alkyl, R3-R4 are independently H, OH, halogen, alkyl, alkoxy, substituted and unsubstituted acyl, substituted alkenyl or substituted alkyl carbonyl, and R5-8 are each hydrogen. Preferably, R1 and R2 are each hydrogen, one of R3 and R4 is methyl and the other is hydrogen and R5-R8 are each hydrogen. In another preferred embodiment, one of R1 and R2 is methyl and the other is hydrogen, one of R3 and R4 is methyl and the other is hydrogen and R5-R8 are each hydrogen. In another preferred embodiment, one of R1 and R2 is methyl and the other is hydrogen, one of R3 and R4 is hydroxymethyl and the other is hydrogen and R5-R8 are each hydrogen. In another preferred embodiment, one of R1 and R2 is methyl and the other is hydrogen, R3 and R4 are each methyl and R5-R8 are each hydrogen.

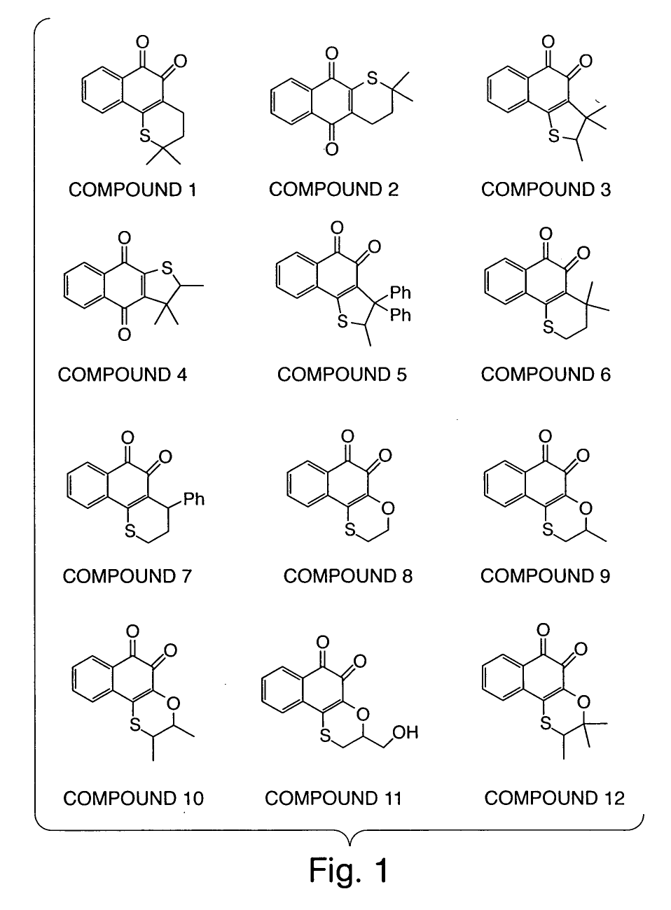

Representative compounds of Formula I, II, III, IV, V are shown in FIG. 1.

The term “alkyl” refers to radicals containing carbon and hydrogen, without unsaturation. Alkyl radicals can be straight or branched. Exemplary alkyl radicals include, without limitation, methyl, ethyl, propyl, isopropyl, hexyl, t-butyl, sec-butyl and the like. A lower alkyl group is a C₁-C₆ alkyl group (e.g., an alkyl group having from one to six carbon atoms in the straight or branched alkyl backbone). Alkyl groups optionally can be substituted. When substituted, alkyl groups may be substituted with up to four substituents, as listed below, at any particular point of attachment (e.g., at any given carbon atom). When the alkyl group is said to be substituted with an alkyl group, this is used interchangeably with “branched alkyl group”. Substitution can be with one or more moieties such as hydroxyl group, carboxylate, oxo, halogen(such as F, Cl, Br, I), haloalkyl (such as CCl₃ or CF₃), alkyloxycarbonyl (—C(O)R), alkylcarbonyloxy (—OCOR), carbamoyl (—NHCOOR— or —OCONHR—), urea (—NHCONHR—), thiol, cyano, nitro, amino, acylamino, C₁-C₆ alkylthio, arylthio, C₁-C₆ alkyl, C₁-C₆ alkoxy, aryloxy, alkylcarbonyloxy, arylcarbonyloxy, C₃-C₆ cycloalkyl, C₃-C₆ cycloalkyloxy, C₂-C₆ alkenyl, C₂-C₆ alkynyl, aryl, aminocarbonyl, C₁-C₆ alkylcarbonyl, C₃-C₆ cycloalkylcarbonyl, heterocyclylcarbonyl, arylcarbonyl, aryloxycarbonyl, C₁-C₆ alkoxycarbonyl, C₃-C₆ cycloalkyloxycarbonyl, heterocyclyloxycarbonyl, C₁-C₆ alkylsulfonyl, arylsulfonyl, a heterocyclyl group, and the like.

The preferred alkyl groups contain 1-6 carbon atoms. Alkylene as used herein refers to a bridging alkyl group of the formula CₙH₂ₙ. Examples include CH₂, —CH₂CH₂—, —CH₂ CH₂CH₂— and the like.

As used herein the term “cycloalkyl” is a species of alkyl containing from 3 to 15 carbon atoms, without alternating or resonating double bonds between carbon atoms. Cycloalkyl species may contain from 1 to 4 rings. Exemplary cycloalkyl groups include cyclopropyl, cyclobutyl, cyclopentyl, cyclohexyl, adamantyl, etc. Exemplary substitutions for cycloalkyl groups include one or more of the following groups: halogen, alkyl, alkoxy, alkyl hydroxy, amino, nitro, cyano, thiol and/or alkylthio.

The term “heterocyclyl” or “heterocycle” refers to a stable non-aromatic 3-7 membered monocyclic heterocyclic ring or 7-11 membered bicyclic heterocyclic ring which is either saturated or unsaturated, and may be fused, spiro or bridged to form additional rings. Each heterocycle consists of one or more carbon atoms and from one to four heteroatoms selected from the group consisting of nitrogen, oxygen and sulfur. A heterocyclyl radical may be attached at any endocyclic atom which results in the creation of a stable structure. Preferred heterocycles include 3-7 membered monocyclic heterocycles (more preferably 5-7-membered monocyclic heterocycles) such as (without limitation) piperidinyl, pyranyl, piperazinyl, morpholinyl, thiamorpholinyl, and tetrahydrofuranyl.

As used herein, the term “alkenyl” refers to unsaturated aliphatic groups analogous in length and possible substitution to the alkyls described above, but that contain at least one carbon-carbon double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups which include oxygen, nitrogen, sulfur or phosphorous atoms replacing one or more hydrocarbon backbone carbons. In certain embodiments, a straight chain or branched chain alkenyl group has six or fewer carbon atoms in its backbone (e.g., C₂-C₆ for straight chain, C₃-C₆ for branched chain). Likewise, cycloalkenyl groups may have from three to eight carbon atoms in their ring structure, and more preferably have five or six carbons in the ring structure. The term “C₂-C₆” includes alkenyl groups containing two to six carbon atoms.

As used herein, the term “alkynyl” refers to unsaturated aliphatic groups analogous in length and possible substitution to the alkyls described above, but that contain at least one carbon-carbon triple bond. For example, the term “alkynyl” includes straight-chain alkynyl groups (e.g., ethynyl, propynyl, butynyl, pentynyl, hexynyl, heptynyl, octynyl, nonynyl, decynyl), branched-chain alkynyl groups (including alkyl or alkenyl substituted alkynyl groups), and cycloalkyl or cycloalkenyl substituted alkynyl groups. The term “alkynyl” further includes alkynyl groups which include oxygen, nitrogen, sulfur or phosphorous atoms replacing one or more hydrocarbon backbone carbons. In certain embodiments, a straight chain or branched chain alkynyl group has six or fewer carbon atoms in its backbone (e.g., C₂-C₆ for straight chain, C₃-C₆ for branched chain.). The term “C₂-C₆” includes alkynyl groups containing two to six carbon atoms.

As used herein, the term “acyl” includes compounds and moieties which contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “aryl” refers to an aromatic carbocyclic or heteroaromatic moiety, having one, two, or three rings. An aryl group may be carbocyclic or may optionally contain from 1-4 heteroatoms (such as nitrogen, sulfur, or oxygen) in the aromatic ring. Exemplary aryl groups include, without limitation, phenyl, naphthyl, pyridyl, pyrimidyl, pyrrolyl, isothiazolyl, triazolyl, tetrazolyl, pyrazolyl, oxazolyl, isooxazolyl, pyrazinyl, pyridazinyl, triazinyl, quinazolinyl, thiazolyl, benzothiophenyl, furanyl, imidazolyl, thiophenyl and the like. An aryl group optionally can be substituted with one or more substituents such as hydroxyl group, halogen, thiol, cyano, nitro, amino, acylamino, C₁-C₆ alkylthio, arylthio, C₁-C₆ alkyl, C₁-C₆ alkoxy, aryloxy, alkylcarbonyloxy, arylcarbonyloxy, C₃-C₆ cycloalkyl, C₃-C₆ cycloalkyloxy, C₂-C₆ alkenyl, C₂-C₆ alkynyl, aryl, carboxylate, aminocarbonyl, C₁-C₆ alkylcarbonyl, C₃-C₆ cycloalkylcarbonyl, heterocyclylcarbonyl, arylcarbonyl, aryloxycarbonyl, C₁-C₆ alkoxycarbonyl, C₃-C₆ cycloalkyloxycarbonyl, heterocyclyloxycarbonyl, aryloxycarbonyl, C₁-C₆ alkoxycarbonyl, C₁-C₆ alkylsulfonyl, arylsulfonyl, a heterocyclyl group, and the like.

As used herein, the term “alkoxy” refers to —O-alkyl groups, wherein alkyl is as defined hereinabove. The alkoxy group is bonded to the main chain, aryl or heteroaryl group through the oxygen bridge. The alkoxy group may be straight chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy.

As used herein, the term “halogen” or “halo” refers to chlorine, bromine, fluorine or iodine.

As used herein, “amine” or “amino” includes compounds where a nitrogen atom is covalently bonded to at least one carbon or heteroatom. “Alkylamino” includes groups and compounds wherein the nitrogen is bound to at least one additional alkyl group. “Dialkylamino” includes groups wherein the nitrogen atom is bound to at least two additional alkyl groups. “Arylamino” and “diarylamino” include groups wherein the nitrogen is bound to at least one or two aryl groups, respectively. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group.

As used herein, “carbonyl” or “carboxy” includes compounds and moieties which contain a carbon connected with a double bond to an oxygen atom. Examples of moieties containing a carbonyl include aldehydes, ketones, carboxylic acids, amides, esters, anhydrides, etc.

As used herein “halogen” or “halo” includes Group VIIa atoms, e.g., fluorine, chlorine, bromine and iodine.

As used herein, the term “salt” is a pharmaceutically acceptable salt and can include acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na, K, Li, alkali earth metal salts such as Mg or Ca, or organic amine salts.

It should be noted that any heteroatom or carbon atom with unsatisfied valences is assumed to have the hydrogen atom to satisfy the valences.

All stereoisomers of the compounds of the instant invention are contemplated, either in admixture or in pure or substantially pure form. The definition of the compounds according to the invention embraces all possible stereoisomers (i.e., the R and S configurations for each asymmetric center) and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization. Furthermore, all geometric isomers, such as E- and Z-configurations at a double bond, are within the scope of the invention unless otherwise stated. Certain compounds of this invention may exist in tautomeric forms. All such tautomeric forms of the compounds are considered to be within the scope of this invention unless otherwise stated.

The present invention also provides pharmaceutical formulations comprising a compound of Formula I, II, III, IV or V in combination with at least one pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, Pa., which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A compound of Formula I, II, III, IV or V is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of a compound of Formula I, II, III, IV or V (as an active ingredient) with standard pharmaceutical carriers or diluents according to conventional procedures (i.e., by producing a pharmaceutical composition of the invention). These procedures may involve mixing, granulating, and compressing or dissolving the ingredients as appropriate to attain the desired preparation. In another embodiment, a therapeutically effective amount of a compound of Formula I, II, III, IV or V is administered in a suitable dosage form without standard pharmaceutical carriers or diluents.

Preferred pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this invention.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients and/or auxiliaries which facilitate processing of the active compounds into preparations that can be used pharmaceutically. Of course, the appropriate formulation is dependent upon the route of administration chosen.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. For treatment of psoriatic conditions, systemic administration (e.g., oral administration), or topical administration to affected areas of the skin, are preferred routes of administration. The dose chosen should be sufficient to constitute effective treatment but not so high as to cause unacceptable side effects. The state of the disease condition (e.g., cancer, psoriasis, and the like) and the health of the patient should preferably be closely monitored during and for a reasonable period after treatment.

The present invention also provides a method for the treatment of cell proliferative disorders in a mammal comprising administering to a mammal in need of such treatment, an therapeutically effective amount of a compound of Formula I, II, III, IV or V. The mammal is preferably a mammal in need of such treatment. The invention further provides the use of a compound of Formula I, II, III, IV or V for the preparation of a medicament useful for the treatment of a cell proliferative disorder. In a preferred embodiment, the invention provides for the treatment of cancer or precancerous conditions in a mammal comprising administering to a mammal in need of such treatment, an therapeutically effective amount of a compound of Formula I, II, III, IV or V.

In a preferred embodiment, an effective amount of a compound of Formula I, II, III, IV or V is used in a method to treat a cell proliferative disorder in a mammal without affecting normal cells of the mammal. Preferably, an therapeutically effective amount of a compound of Formula I, II, III, IV or V is used in a method for treating cancer in a mammal by inducing apoptosis in cancer cells without affecting normal cells in the mammal. In another preferred embodiment, administration of a therapeutically effective amount of a compound of Formula I, II, III, IV or V induces sustained (non-transient) activity (e.g. elevation of the level) of a member of the E2F family of transcription factors (including but not limited to E2F1, E2F2 or E2F3) in abnormally proliferating cells without affecting E2F activity (e.g. E2F levels) in normal cells. Preferably, administration induces sustained E2F activity (e.g. elevation of E2F levels) in cancer cells without affecting E2F activity (e.g. E2F levels) in normal cells. Methods of measuring induction of E2F activity and elevation of E2F levels are as shown in Li et al., (2003) Proc Natl Acad Sci USA. 100(5): 2674-8. In another preferred embodiment, administration of a therapeutically effective amount of a compound of Formula I, II, III, IV or V induces apoptosis in abnormally proliferating cells without inducing apoptosis in normal cells.

The invention also provides a method of protecting against a cell proliferative disorder in a mammal by administering an therapeutically effective amount of a compound of Formula I, II, III, IV or V to a mammal. The invention also provides the use of a compound of Formula I, II, III, IV or V for the preparation of a medicament useful for the prevention of a cell proliferative disorder. In a preferred embodiment, the invention provides for the prevention of cancer in a mammal comprising administering to a mammal in need of such treatment, an therapeutically effective amount of a compound of Formula I, II, III, IV or V.

In another embodiment, the present invention provides a method of treating or protecting against a cell proliferative disorder in a mammal by administering an therapeutically effective amount of a compound of Formula V′:

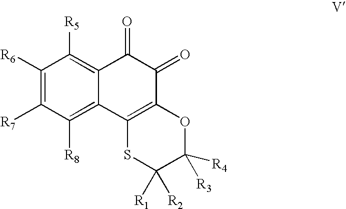

or pharmaceutically acceptable salts thereof or a regioisomeric mixture thereof, wherein

R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R1 or R2 and one of R3 or R4 form a fused ring, wherein the ring has 4-8 ring members;

R5-R8 are each, independently, hydrogen, hydroxyl, halogen, substituted or unsubstituted alkyl, substituted or unsubstituted alkoxy, nitro, cyano or amide; and

n is an integer from 1 to 10.

Preferably, an therapeutically effective amount of a compound of Formula V′ is used in a method for treating cancer in a mammal by inducing apoptosis in cancer cells without affecting normal cells in the mammal. In another preferred embodiment, administration of a therapeutically effective amount of a compound of Formula V′ induces sustained (non-transient) activity (e.g. elevation of the level) of a member of the E2F family of transcription factors (including but not limited to E2F1, E2F2 or E2F3) in abnormally proliferating cells without affecting E2F activity (e.g. E2F levels) in normal cells. Preferably, administration induces sustained elevation of E2F activity (e.g. elevation of E2F levels) in cancer cells without affecting E2F activity (e.g. E2F levels) in normal cells. In another preferred embodiment, administration of a therapeutically effective amount of a compound of Formula V′ induces apoptosis in abnormally proliferating cells without inducing apoptosis in normal cells.

The compounds of the invention are preferably administered in the form of pharmaceutical compositions, e.g., as described herein.

The mammal can be e.g., any mammal, e.g., a human, a primate, mouse, rat, dog, cat, cow, horse, pig. In a preferred embodiment, the mammal is a human.

As used herein, the term “cell proliferative disorder” refers to conditions in which the unregulated and/or abnormal growth of cells can lead to the development of an unwanted condition or disease, which can be cancerous or non-cancerous, for example a psoriatic condition. As used herein, the term “psoriatic condition” refers to disorders involving keratinocyte hyperproliferation, inflammatory cell infiltration, and cytokine alteration.

In a preferred embodiment, the cell proliferation disorder is cancer. As used herein, the term “cancer” includes solid tumors, such as lung, breast, colon, ovarian, prostate, malignant melanoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS.

In addition to psoriatic conditions, the types of proliferative diseases which may be treated using the compositions of the present invention are epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses and the like. In one embodiment, proliferative diseases include dysplasias and disorders of the like.

The invention also provides methods for the synthesis of the compounds of Formula I, II, III, IV or V. In a preferred embodiment, the present invention provides a method for the synthesis of compounds according to scheme 1.

In one embodiment, the present invention provides a method for the synthesis of a compound of Formula I comprising reacting a compound having the Formula A:

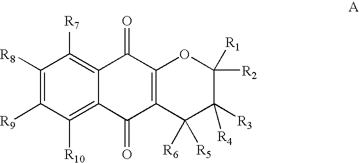

wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R7-R10 are each, independently, hydrogen, hydroxyl, halogen, substituted or unsubstituted alkyl, substituted or unsubstituted alkoxy, nitro, cyano or amide; and

n is an integer from 0 to 10,

with sodium hydrosulfide to exchange oxygen for sulfur and forming the di-thione of the compound of Formula A;

and,

reacting said di-thione of the compound of Formula A with a strong acid to form a compound of Formula I.

In a preferred embodiment, compound formed of Formula I is compound 1. In another preferred embodiment, the strong acid is concentrated sulfuric acid.

In another embodiment, the present invention provides a method for the synthesis of a compound of Formula II comprising reacting a compound having the Formula B:

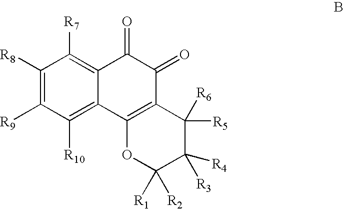

wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R7-R10 are each, independently, hydrogen, hydroxyl, halogen, substituted or unsubstituted alkyl, substituted or unsubstituted alkoxy, nitro, cyano or amide; and

n is an integer from 0 to 10,

wherein when R1 and R2 are both methyl, R3 and R4 are both H, and one of R5 and R6 is OH and the other H, R7 is not methyl or methoxy and R10 is not methyl,

with sodium disulfide to exchange oxygen for sulfur and forming the di-thione of the compound of Formula B;

and,

reacting said di-thione of the compound of Formula B with a strong acid to form a compound of Formula II.

In a preferred embodiment, compound formed of Formula II is compound 2. In another preferred embodiment, the strong acid is concentrated sulfuric acid.

In another embodiment, the present invention provides a method for synthesizing a compound of formula III, comprising reacting a compound having the Formula C:

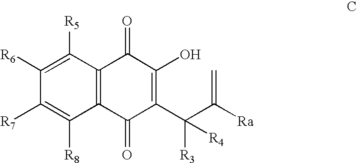

wherein Ra is selected from R1 and R2;

R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R5-R8 are each, independently, hydrogen, hydroxyl, halogen, substituted or unsubstituted alkyl, substituted or unsubstituted alkoxy, nitro, cyano or amide; n is an integer from 0 to 10

with sodium hydrosulfide to form the di-thione of the compound of Formula C,

and,

treating said di-thione of compound of Formula C with a strong acid to form a compound of Formula III.

In a preferred embodiment, compound formed of Formula III is compound 3. In another preferred embodiment, the strong acid is concentrated sulfuric acid.

In another embodiment, the present invention provides a method for synthesizing a compound of formula IV reacting a compound having the Formula D:

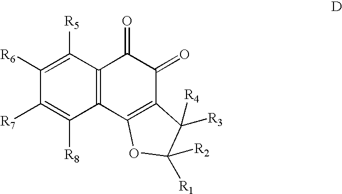

R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;

R5-R8 are each, independently, hydrogen, hydroxyl, halogen, substituted or unsubstituted alkyl, substituted or unsubstituted alkoxy, nitro, cyano or amide; and

n is an integer from 0 to 10,

wherein:

when R1 and R2 are both methyl, one of R3 and R4 is not methyl and the other H, and each of R5, R6, R7 and R8 is not H;

when R1 and R2 are both methyl, at least one of R3, R4, R5, R6, R7 and R8 is not H;

when R1 and R2 are both phenyl, at least one of R3, R4, R5, R6, R7 and R8 is not H;

when one of R1 and R2 is phenyl and the other is H, at least one of R3, R4, R5, R6, R7 and R8 is not H;

when one of R1 and R2 is phenyl and the other is methyl, at least one of R3, R4, R5, R6, R7 and R8 is not H;

when one of R1 and R2 is methyl and the other is H and R5, R6, R7 and R8 are H, at least one of R3 and R4 is not OH and the other H;

when one of R3 and R4 are carboethoxy the other is H; and

R1, R2, R3, R4, R5, R6, R7 and R8 are not each H,

with sodium hydrosulfide to form the di-thione of the compound of Formula D,

and,

treating said di-thione of the compound of Formula D with a strong acid to form a compound of Formula IV.

In a preferred embodiment, compound formed of Formula IV is compound 4. In another preferred embodiment, the strong acid is concentrated sulfuric acid.

In a preferred embodiment, the present invention provides a method for the synthesis of compounds according to scheme 2. In one embodiment, the present invention provides a method for the synthesis of a compound of Formula I comprising eacting a compound having the Formula E:

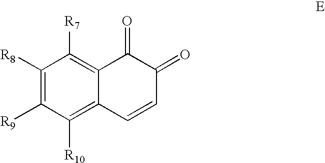

wherein R7-R10 are each, independently, hydrogen, hydroxyl, halogen, substituted or unsubstituted alkyl, substituted or unsubstituted alkoxy, nitro, cyano or amide,

with a branched allylthiol having the formula:

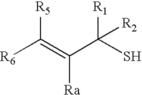

wherein Ra is selected from R3 and R4;

R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; and, and n is an integer from 0 to 10,

in the presence of a weak base to form the sulfide intermediate having the formula:

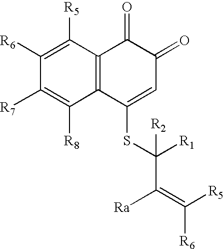

and,

treating said sulfide intermediate with a strong acid to form the a compound of Formula I.

In a preferred embodiment, compound formed of Formula I is compound 5. In another preferred embodiment, the strong acid is concentrated sulfuric acid and the weak base is triethylamine.

In another preferred embodiment, the present invention provides a method for the synthesis of a compound of Formula III comprising reacting a compound having the Formula F:

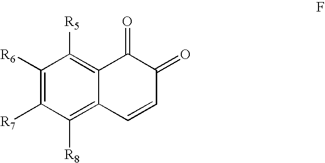

wherein R5-R8 are each, independently, hydrogen, hydroxyl, halogen, substituted or unsubstituted alkyl, substituted or unsubstituted alkoxy, nitro, cyano or amide,

with 2-hydroxyalkylthiol having the formula:

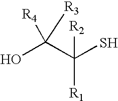

wherein R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxycarbonyl, —(CH₂)ₙ-amino, —(CH₂)ₙ-aryl, —(CH₂)ₙ-heterocycle, —(CH₂)ₙ-phenyl, aryl, heterocycle, and phenyl; and n is an integer from 1 to 10

in the presence of a weak base to form a sulfide intermediate having the formula:

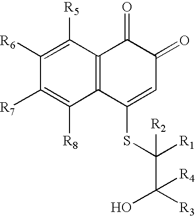

and,

treating said sulfide intermediate with a strong acid to form the a compound of Formula III.

In a preferred embodiment, compounds formed of Formula III are compounds 6 and 7. In another preferred embodiment, the strong acid is concentrated sulfuric acid and the weak base is triethylamine.

In another preferred embodiment, the present invention provides a method for synthesizing a compound of Formula V, comprising reacting a compound having the Formula G:

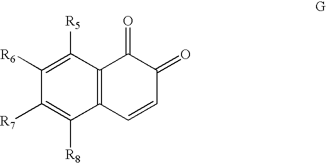

wherein R5-R8 are each, independently, hydrogen, hydroxyl, halogen, substituted or unsubstituted alkyl, substituted or unsubstituted alkoxy, nitro, cyano or amide,

with a 2-hydroxyalcohol having the formula:

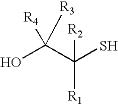

wherein R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; n is an integer from 1 to 10,

to form a sulfide having the formula:

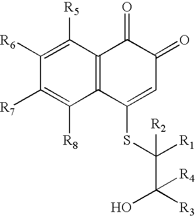

and,

treating said sulfide with strong acid and exposing the reaction to an oxidizing agent to form a compound of Formula V.

In a preferred embodiment, compounds formed of Formula V are compounds 8, 9, 10, 11 and 12. In another preferred embodiment, the strong acid is concentrated sulfuric acid or trifluoroacetic acid. In another preferred embodiment, the oxidizing agent is oxygen gas. Preferably, the oxygen gas is the air.

The invention will be further described in the following examples, which do not limit the scope of the invention described in the claims.

### EXAMPLES

### General

Compounds of the invention can be prepared in a variety of ways, some of which are known in the art. In general, the compounds of the present invention can be prepared from commercially available starting materials, compounds known in the literature, or from readily-prepared intermediates, by employing standard synthetic methods and procedures known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}.; John Wiley & Sons: New York, 1999 are useful and recognized reference textbooks of organic synthesis known to those in the art. The following descriptions of synthetic methods are designed to illustrate, but not limit, general procedures for the preparation of compounds of the invention.

Chromatography refers to column chromatography on silica gel using CH₂Cl₂ as eluant, unless otherwise stated. ¹H NMR spectra were recorded at 400 MHz using tetramethylsilane as an internal standard.

### Example 1

### Synthesis of Compounds of Formula I, II, III, IV or V

Compounds 1, 2, 3 and 4 of the current invention were synthesized by a two step reaction process, starting from appropriately-substituted naphthoquinone analogs with O-containing hetero-rings. The two steps are: 1) exchange of the oxygen atoms on the carbonyl groups of the naphthoquinone molecule with sulfur atoms by treatment with sodium hydrosulfide; and 2) formation of dihydrothiopyran or dihydrothiophene hetero-rings by in situ addition of concentrated sulfuric acid. The first reaction step is a common nucleophilic reaction wherein hydrosulfide anions attack the carbonyl groups to form sulfur-substituted carbonyl groups; the yield of this step is determined by solvent polarity and the stability of the substituted carbonyl group in the solvent. The second reaction step is based on the observation that β-lapachone (3,4-dihydro-2,2-dimethyl-2H-naphtho(1,2-b)pyran-5,6-dione) and α-lapachone (3,4-dihydro-2,2-dimethyl-2H-naphtho[2,3-b]pyran-5,10-dione) can be converted to each other in concentrated sulfuric acid. The second reaction step involves formation of a carbon cation intermediate at position 2 (see structure of β-lapachone above), which results from breakage of the ether linkage (for example, when α-lapachone, β-lapachone or dunione are the starting material) or protonation of the alkene group (for example, when lapachol analog is the starting material). The yield of this step is determined by the stability of the carbon cation, and the ratio of the two isoforms (α and β) can be determined to some extent by the temperature of the sulfuric acid solution. Any substitution at position 2 stabilizing the carbon cation should improve yield of this reaction step. The desired isoforms can be isolated and purified by extraction and column chromatography (silica gel).

The process used for the preparation of compound 1 (3,4-dihydro-2,2-dimethyl-2H-naphtho[1,2-b]thiopyran-5,6-dione), compound 2 (3,4-dihydro-2,2-dimethyl-2H-naphtho[2,3-b]thiopyran-5,10-dione), compound 3 (2,3,3-trimethyl-2H-3H-naphtho[1,2-b]thiophene-4,5-dione), and compound 4 (2,3,3-trimethyl-2H-3H-naphtho[1,2-b]thiopyran-4,9-dione) was as follows (also, see Scheme 1 below): In a dried 250 ml round-bottom flask, 1.5 g (6.2 mmol) of α-Lapachone for compound 1 (or β-Lapachone, Lapachol analog, or Dunnione for compounds 2, 3 or 4, respectively) was dissolved in 20 ml of anhydrous THF under an argon atmosphere. Then 1.5 g (19.2 mmol) of dry sodium hydrosulfide was added into the solution, and the resultant mixture was stirred vigorously at room temperature under an argon atmosphere for 1 hour. 4.5 ml (18 mmol) of 4 M HCl solution in 1,4-dioxane was added into the mixture and the mixture was stirred for another 4 hours at the same conditions. For synthesis of compound 1 and compound 3, the reaction mixture was cooled in an ice bath, and then 100 ml of concentrated sulfuric acid was added, and the resultant mixture was stirred at 0° C. under an argon atmosphere for 20 min. For the synthesis of compound 2 and compound 4, 100 ml of concentrated sulfuric acid was added directly at room temperature (the reaction between sulfuric acid and excess sodium hydrosulfide heated up the reaction mixture) and the resultant mixture was stirred at room temperature under an argon atmosphere for 20 min. The reaction was stopped by pouring the mixture into 400 ml of ice water, and the resultant mixture was extracted with 2×150 ml of dichloromethane. The organic phases were pooled and washed successively with water (300 ml), 5% aqueous sodium bicarbonate (300 ml) and 1% aqueous sodium chloride (300 ml), and then dried with sodium sulfate (20 grams). After filtration, the filtrate was evaporated to dryness by Rotovap. The residue was dissolved in 20 ml of dichloromethane/hexane (1:1), and loaded on to a silica gel column. Dichloromethane was used to elute compound 1 and compound 3, and dichloromethane/hexane (2:1) was used to elute compound 2 and compound 4. One major impurity accompanied each product. The obtained impurities were β-Lapachone, α-Lapachone, dunnione and 2,3,3-trimethyl-2H-3H-naphtho[2,3-b]furan4,9-dione (α-dunnione), respectively, for compound 1, compound 2, compound 3 and compound 4 reaction mixtures. NMR and mass spectra (MS) analyses of the products were performed by Spectral Data Service, Inc., 818 Pioneer St., Champaign, Ill. 61820 and HT Laboratories, 9823 Pacific Height Blvd., Suite F, San Diego, Calif. 92121. NMR and MS analysis confirm the structure of compound 1, compound 2, and compound 3, as shown in Table 1. α-dunnione strongly suggests the isolated compound has the structure shown for compound 4.

The overall yields of compounds produced by this synthesis method were between 2% and 10%. As mentioned above, yield depends upon substitution at position 2 of the starting naphthoquinone, with the tertiary structure at position 2 in β-lapachone providing a more stable intermediate and thus higher yields than the secondary carbon at position 2 in dunnione. Since product yield also depends on solvent choice, solvents other than tetrahydrofuran (THF) may be used to achieve higher yields.

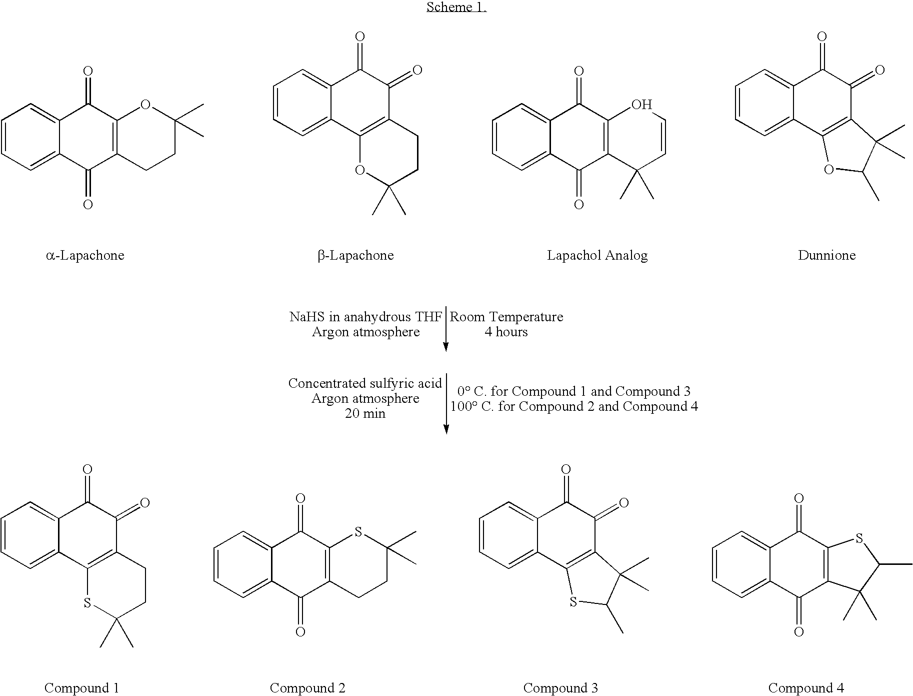

Compounds 5, 6 and 7 were synthesized by a two step reaction process. The two steps are: (1) arylation of 2-hydroxyalkylthiol or branched allylthiol by reacting with 1,2-naphthoquinone in acetonitrile in the presence of small amount of triethylamine; (2) formation of dihydrothiopyran or dihydrothiophene hetero-rings by treatment with concentrated sulfuric acid. The first reaction step is a well known quinone arylation reaction. The instant method differs from the method reported by Mackenzie et al., (1986) J. Chem. Soc. Perkin Tran. I, 2233-2241), as the current method is the first example of the use of a weak base (i.e. triethylamine) which reduced reaction time and enhanced product yield. The second reaction step is a novel reaction involving a cation intermediate which is formed by protonization of tertiary hydroxyl or branched allyl. Since the cation intermediate is more difficult to form from protonization of a secondary hydroxyl, the semidehydrothioxane hetero-rings were formed from 2-hydroxyalkylthiol arylation product when treated with concentrated sulfuric acid as determined previously.

The process used for the preparation of compound 5 (2-methyl-3,3-diphenyl-2H-3H-naphtho[1,2-b]thiophene4,5-dione), compound 6 (3,4-dihydro4,4-dimethyl-2H-naphtho[1,2-b]thiopyran-5,6-dione), and compound 7 (3,4-dihydro-4-phenyl-2H-naphtho[1,2-b]thiopyran-5,6-dione) is as follows (also, see Scheme 2 below): In a dried 250 ml round-bottom flask, 10 mmol of 1,1-diphenyl-2-Mercapto-1-propanol (or 3-methyl-1-mercapto-2-butene, or Cinnamyl thiol for compound 5, compound 6 or compound 7, respectively) was dissolved in 100 ml of acetonitrile, then 0.5 ml of triethylamine (TEA) and 1.58 g (10 mmol) of 1,2-naphthoquinone were added. The mixture was stirred at room temperature for 2 hrs for compounds with a primary thiol or overnight for compounds with a secondary thiol, then evaporated to dryness. 20 ml of anhydrous toluene was added into the residue, and evaporated again to dryness. The residue was dissolved in 15 ml of methylene chloride and loaded onto a silica gel column. The intermediate product 4-(2-hydroxyalkylsulfanyl or branched allylsulfanyl)-1,2-naphthoquinone was eluted by ethyl acetate/hexane (1:1). The yield of the intermediate product was at range of 10% to 80%, high yield for primary thiol and low yield for secondary thiol.

The purified intermediate product 4-(2-hydroxyalkylsulfanyl or branched allylsulfanyl)-1,2-naphthoquinone was dissolved in 50 times by weight of concentrated sulfuric acid and the mixture was placed at room temperature for 30 min, and then poured into 10 times volume of ice-water, and the resulting mixture was extracted with methylene chloride. The organic phase was washed with water and dried with sodium sulfate. After filtration, the filtrate was evaporated into dryness, and then dissolved in 10 ml of methylene chloride and loaded onto a silica gel column. The final pure product was eluted with methylene chloride/ethyl acetate (10:0 to 10:2). NMR and MS analyses of the products were performed as described above. Results are shown in Table 1.

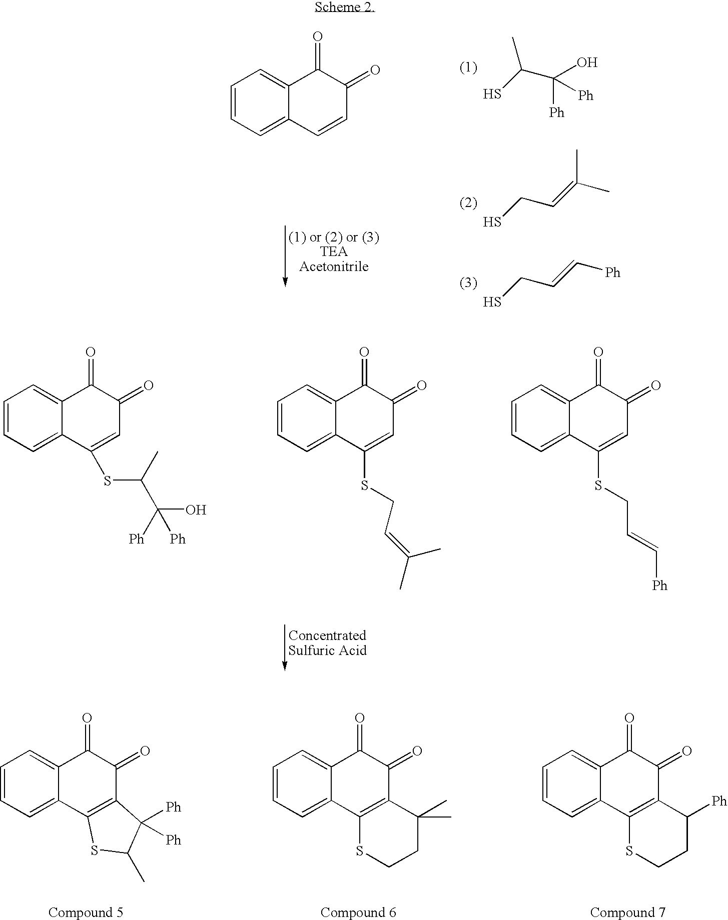

Compounds 8, 9, 10, 11 and 12 were synthesized by a two step reaction process. The two steps are: (1) arylation of 2-hydroxyalkylthiol by reacting with 1,2-naphthoquinone in acetonitrile in the presence of trace amount of triethylamine; (2) formation of semidehydrothioxane hetero-rings by treatment with concentrated sulfuric acid (or trifluoroacetic acid) and exposure to air. The first reaction step is a well known quinone arylation reaction. The instant method differs from the method reported by Mackenzie et al., (1986) J. Chem. Soc. Perkin Tran. I, 2233-2241 as the current method used a weak base (i.e. triethylamine) which reduced reaction time and enhanced product yield. The second reaction step is novel reaction, and the mechanism is not yet clear. The 2-hydroxyalkylthiol arylation product as 4-(2-hydroxyethylsulfanyl)-1,2-naphthoquinone is very stable at room temperature when exposed to the air, which is contrary to previous studies (Sugiyama et al., (1999) Drug Metabolism and Disposition, 27(1): 60-67), and forms semidehydrothioxane hetero-rings only when treated with strong acid like concentrated sulfuric acid or trifluoroacetic acid.

The process used for the preparation of compound 8 (11,12-dehydro-naphtho[1,2-b]thioxane-9,10-dione), compound 9 (2-methyl-11,12-dehydro-naphtho[1,2-b]thioxane-9,10-dione), compound 10 (2,3-dimethyl-11,12-dehydro-naphtho[1,2-b]thioxane-9,10-dione), compound 11 (2-hydroxymethyl-11,12-dehydro-naphtho[1,2-b]thioxane-9,10-dione), and compound 12 (2,2,3-trimethyl-11,12-dehydro-naphtho[1,2-b]thioxane-9,10-dione) is as follows (also, see Scheme 3 below): In a dried 250 ml round-bottom flask, 10 mmol of 2-Mercaptoethanol (or 1-Mercapto-2-Propanol, 3-Mercapto-2-Butanol, 3-Mercapto-1,2-Propanediol, 2-Methyl-3-Mercapto-2-Butanol, other 2-hydroxyalkylthiol) was dissolved in 100 ml of acetonitrile, then 0.5 ml of triethylamine and 1.58 g (10 mmol) of 1,2-naphthoquinone were added. The mixture was stirred at room temperature for 2 hrs for primary thiol or overnight for secondary thiol, then evaporated to dryness. 20 ml of anhydrous toluene was added into the residue, and evaporated again to dryness. The residue was dissolved in 15 ml of methylene chloride and loaded onto a silica gel column. The intermediate product 4-(2-hydroxyalkylsulfanyl)-1,2-naphthoquinone was eluted by ethyl acetate/hexane (9:1 to 1:1, depend on the polarity of the 2-hydroxyalkylthiol). The yield of the intermediate product was at range of 10% to 80%, high yield for primary thiol and low yield for secondary thiol.

The purified intermediate product 4-(2-hydroxyalkylsulfanyl)-1,2-naphthoquinone was dissolved in 50 times weight of trifluoroacetic acid (or concentrated sulfuric acid) and the mixture was placed at room temperature for 30 min. To trifluoroacetic acid solution, evaporation and co-evaporation with toluene was applied to remove trifluoroacetic acid. However, the concentrated sulfuric acid solution was poured into 10 times volume of ice-water, and the resulting mixture was extracted with methylene chloride. The organic phase was then washed with water and dried with sodium sulfate. After filtration, the filtrate was evaporated into dryness. In both case, the crude product residue was dissolved in 10 ml of methylene chloride and loaded onto a silica gel column. The final pure product was eluted with methylene chloride/ethyl acetate (10:0 to 10:2). NMR and MS analyses of the products were performed as described above. Results are shown in Table 1.

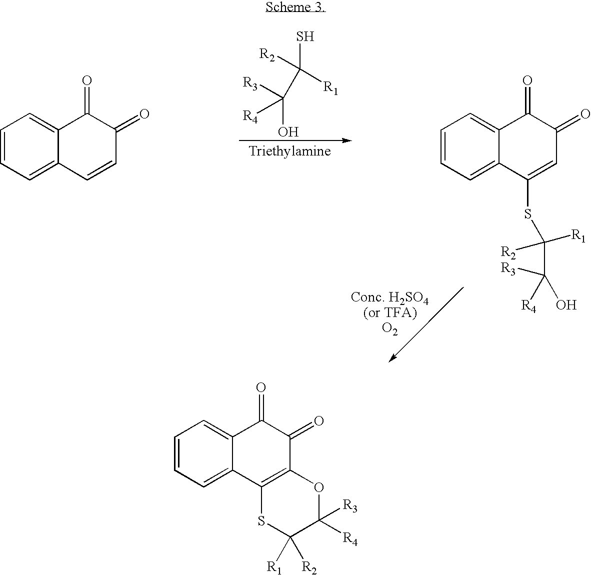

TABLE 1

| | | Theoretic | Mass Spectra |
|---|---|---|---|
| Compound | NMR data | M.W. | M.W. |
| Compound 1 | 1.45 (s, 6-CH₃), 1.91 (t, J=7, 2H-3), 2.83 (t, | 258 | 258 |
| | J=7, 2H-4), 7.48 (t, J=8, 1H), 7.63 (t, J=8, 1H), | | |
| | 7.79 (d, J=8, 1H), 8.11 (d, J=8, 1H) | | |
| Compound 2 | 1.22 (s, 6-CH₃), 1.62 (t, J=7, 2H-3), 2.65 (t, | 258 | 258 |
| | J=7, 2H-4), 7.42 (t, J=7, 1H), 7.52 (t, J=7, 1H), | | |
| | 8.06 (d, J=8, 1H), 8.13 (d, J=8, 1H) | | |
| Compound 3 | 1.21 (d, J=7, 3-CH₃), 1.39 (s, 3-CH₃), 1.59 (s, | 258 | 258 |
| | 3-CH₃), 3.98 (q, J=7, 1H-2), 7.47 (d, J=8, | | |
| | 1H), 7.55 (t, J=8, 1H), 7.64 (t, J=8, 1H), | | |
| | 8.09 (d, J=8, 1H) | | |
| Compound 4 | N/A | 258 | N/A |
| Compound 5 | 1.09 (d, J=7, 3-CH₃), 4.36 (q, J=7, 1H-2), | 382 | 382 |
| | 7.1-8.08 (m, 14H) | | |
| Compound 6 | 1.43 (s, 6-CH₃), 1.98 (t, J=7, 2H-3), 3.10 (t, | 258 | 258 |
| | 2H-2), 7.47 (t, J=8, 1H), 7.64 (t, J=8, 1H), | | |
| | 7.88 (d, J=8, 1H), 8.07 (d, J=8, 1H) | | |
| Compound 7 | 2.08-2.17 (m, 1H-3), 2.34-2.39 (m, 1H-3), | 306 | 306 |
| | 2.96-3.01 (m, 2H-2), 4.66-4.68 (m, 1H-4), | | |
| | 7.17-7.30 (m, 5H-4-phenyl), 7.54 (t, J=8, 1H), | | |
| | 7.7 (t, J=8, 1H), 7.94 (d, J=8, 1H), 8.14 (d, J=8, 1H) | | |
| Compound 8 | 3.16 (t, J=5, 2H-3), 4.69 (t, J=5, 2H-2), 7.48 (t, | 232 | 232 |
| | J=7, 1H), 7.64 (t, J=8, 1H), 7.72 (d, J=8, 1H), | | |
| | 8.04 (d, J=7, 1H) | | |
| Compound 9 | 1.63 (d, J=6, —CH₃), 2.92 (q, J=7, 1H-3), | 246 | 246 |
| | 3.09 (q, J=2, 10, 1H-3), 4.65-4.70 (m, 1H-2), | | |
| | 7.47 (t, J=7, 1H), 7.64 (t, J=7, 1H), 7.73 (d, | | |
| | J=8, 1H), 8.04 (d, J=8, 1H) | | |
| Compound 10 | 1.37 (d, J=7, 3-CH₃), 1.50 (d, J=7, 3-CH₃), | 260 | 260 |
| | 3.34 (q, J=6, 1H-3), 4.77 (q, J=6, 1H-2), | | |
| | 7.47 (t, J=7, 1H), 7.64 (t, J=7, 1H), 7.74 (d, | | |
| | J=8, 1H), 8.04 (d, J=8, 1H) | | |
| Compound 11 | 2.96-3.01 (m, 1H-3), 3.21-3.25 (m, 1H-3), | 262 | 262 |
| | 3.78 (t, J=6, 2H), 4.42-4.46 (m, 1H-2), 5.27 (t, | | |
| | J=6, 1H), 7.55 (t, J=7, 1H), 7.75 (t, J=7, 1H), | | |
| | 7.82 (d, J=8, 1H), 7.89 (d, J=8, 1H) | | |
| Compound 12 | 1.38 (d, J=7, 3-CH₃), 1.48 (s, 3-CH₃), 1.63 (s, | 274 | 274 |
| | 3-CH₃), 3.18 (q, J=7, 1H-3), 7.46 (t, J=7, 1H), | | |
| | 7.63 (t, J=7, 1H), 7.73 (d, J=7, 1H),), 8.04 (d, | | |
| | J=8, 1H) | | |

N/A: Data not available

### Example 2

### Antiproliferative Activity of Compounds of Formula I, II, III, IV or V

Compounds of the present invention have demonstrated potent antiproliferative activity against a variety of cancer cell lines, including SK-OV-3 and OVCAR-3 human ovarian carcinoma cells; SW-480, HT-29, DLD1 and HCT-116 human colon carcinoma cells; MCF-7 and MDA-MB-231 human breast carcinoma cells; MIA PACA-2 and BXPC-3 human pancreatic carcinoma cells; NCI-H226 and A549 human lung carcinoma cells; and DU-145 and PC-3 human prostate cancer cells. Since β-lapachone induces apoptosis only in cancer cell lines and not in normal cells (Li et al., (2003) Proc Natl Acad Sci USA. 100(5): 2674-8), the present compounds were also tested in a panel of normal cell lines from a variety of tissues including NCM 460 normal colonic epithelial cells and MCF 10A normal breast epithelial cells.

Table 2 shows the concentrations of the compounds required to inhibit 50% of cell growth (IC₅₀). As shown in Table 2, IC₅₀ values in the low micromolar range and below were obtained for several of these compounds in all cancer cell lines tested.

Another effect of the compounds of the present invention is the induction or elevation of activity (e.g. elevation of the level) of a member of the E2F family of transcription factors. Studies have shown that β-lapachone induces sustained E2F activity (e.g. elevation of E2F levels) in nuclei of cancer cells but not in normal cells, resulting in the arrest of cancer cells in G1 and/or S phase. Several compounds of the present invention were effective in sustaining E2F activity (e.g. elevation of E2F levels), thus causing G1 and/or S phase arrest. Furthermore, the compounds of the present invention have no significant toxic effects on normal cells (See, Table 2).

Cell proliferation assays were performed as described previously (Müller et al., (1996) J. Med. Chem. 39: 3132-3138; Müller et al., (1994) J. Med. Chem. 37: 1660-1669). More specifically, exponentially growing cells were seeded at 1,000 cells per well in six-well plates and allowed to attach for 24 h. Compounds of the present invention, or β-lapachone, or Dunnione, were solubilized in DMSO and were added to the wells in micromolar concentrations. Control wells were treated with equivalent volumes of DMSO. After 4 h, the supernatant was removed and fresh medium was added. Cultures were observed daily for 10-15 days and then were fixed and stained. Colonies of greater than 30 cells were scored as survivors.

The assays of the present invention as shown in Table 2 and methods of measuring induction of E2F activity and elevation of E2F levels can be carried out following the descriptions found in Li et al., (2003) Proc Natl Acad Sci USA. 100(5): 2674-8 and U.S. Patent Application Publication No.2002/0169135, both incorporated herein by reference in their entireties.

The antiproliferative activity of the present synthetic lapachone derivative compounds suggests that compounds of the present invention may be expected to show wide anticancer activity. For example, the compounds of the invention are effective for treating cancers such as breast cancer, leukemia, lung cancer, ovarian cancer, brain cancer, liver cancer, pancreatic cancer, prostate cancer, and colorectal cancer. These treatments are accomplished utilizing the present lapachone derivative compounds (Formula I, II, III, IV or V), alone or in combination with, other chemotherapy agents or with radiation therapy. In a preferred embodiment the compounds of the present invention are used for the prevention or treatment of hyperproliferative disorders and cancer (e.g., as a preventative drug) by preventing hyperproliferative or cancer cell formation.

The results of experiments with β-lapachone and similar chemical compounds have shown that the compounds of the present invention have a strong apoptotic effect on a variety of human cancer cells and that they can inhibit growth of other human cancer cells. They can be applied in many of the well-known methods currently used for chemotherapeutic treatment. For example, they may be injected directly into tumors, injected into the blood stream or body cavities or taken orally or applied through the skin with patches. The dose chosen should be sufficient to constitute effective treatment but not so high as to cause unacceptable side effects. The state of the cancer and the health of the patient should preferably be closely monitored during and for a reasonable period after treatment.

TABLE 2

| | | | Average IC₅₀ | | | | | | |
|---|---|---|---|---|---|---|---|---|---|
| | | | | Cancer Cell Lines | | | | | |
| Compound | | | | Colon | Colon | Breast | Pancr. | Lung | Colon |
| No. | Structure | Name | MW | DLD1 | SW480 | MCF7 | PaCa-2 | A549 | HT-29 |
| N/A | 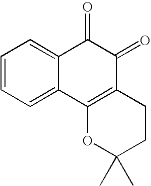 | 3,4-dihydro-2,2-dimethyl- 2H-naphtho[1,2-b]pyran-5,6 dione (β-Lapachone) | 242 | 3.6 | 3.3 | 1.0 | 1.6 | 1.8 | 2.1 |
| N/A | 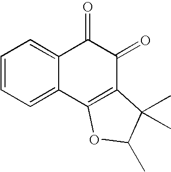 | 2,3,3-trimethyl-2,3,4,5- tetrahydro-naphtho(2,3- b)dihydrofuran-6,7-dione (Dunnione) | 242 | 2.8 | 2.8 | 1.0 | — | — | — |
| 1 | 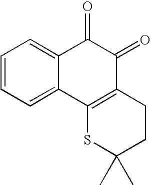 | 3,4-dihydro-2,2-dimethyl- 2H-naphtho[1,2-b]thiopyran- 5,6-dione | 258 | 2.0 | 1.5 | 1.0 | 1.5 | 1.7 | 1.8 |
| 2 | 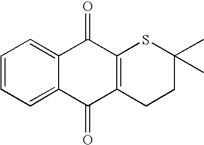 | 3,4-dihydro-2,2-dimethyl- 2H-naphtho[2,3-b]thiopyran- 5,10-dione | 258 | 32.0 | 32.0 | 16.0 | — | — | — |
| 3 | 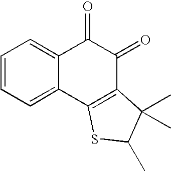 | 2,3,3-trimethyl-2H-3H- naphtho[1,2-b]thiophene- 4,5-dione | 258 | 0.9 | 0.9 | 1.0 | — | — | 1.5 |
| 5 | 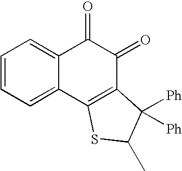 | 2-methyl-3,3-diphenyl-2H- 3H-naphtho[1,2- b]thiophene-4,5-dione | 382 | — | — | — | 1.3 | 2.3 | 1.7 |
| 6 | 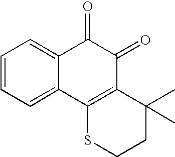 | 3,4-dihydro-4,4-dimethyl- 2H-naphtho[1,2-b]thiopyran 5,6-dione | 258 | — | — | — | 1.2 | 1.6 | 1.6 |
| 7 | 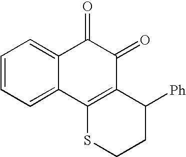 | 3,4-dihydro-4-phenyl-2H- naphtho[1,2-b]thiopyran-5,6 dione | 306 | — | — | — | 2.3 | 2.0 | 2.2 |
| 9 | 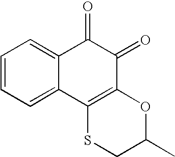 | 2-methyl-11,12-dehydro- naphtho[1,2-b]thioxane- 9,10-dione | 230 | — | 1.5 | — | 1.4 | 2.0 | 1.5 |
| 10 | 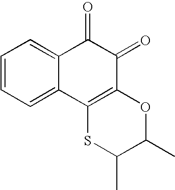 | 2,3-dimethyl-11,12-dehydro naphtho[1,2-b]thioxane- 9,10-dione | 244 | — | 1.0 | — | 2.0 | 1.8 | 2.0 |
| 12 | 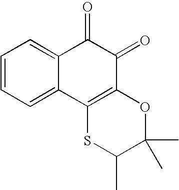 | 2,2,3-trimethyl-11,12- dehydro-naphtho[1,2- b]thioxane-9,10-dione | 274 | — | — | — | 2.2 | 3.1 | 4.0 |

| | | | Normal Cell Lines | | E2F1 Induction | | |
|---|---|---|---|---|---|---|---|
| Compound | | | Colon | Breast | E2F1 | E2F1 | E2F1 |
| No. | Structure | Name | NCM460 | MCF10A | SW480 | HT-29 | PANC1 |
| N/A | 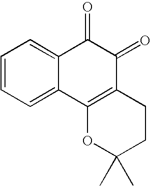 | 3,4-dihydro-2,2-dimethyl- 2H-naphtho[1,2-b]pyran-5,6 dione (β-Lapachone) | 8.1 | 5.8 | + | + | + |
| N/A | 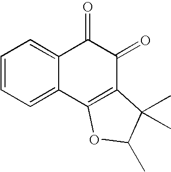 | 2,3,3-trimethyl-2,3,4,5- tetrahydro-naphtho(2,3- b)dihydrofuran-6,7-dione (Dunnione) | 4.0 | — | — | — | — |
| 1 | 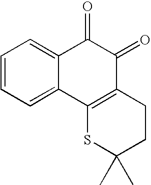 | 3,4-dihydro-2,2-dimethyl- 2H-naphtho[1,2-b]thiopyran- 5,6-dione | 7.5 | 6.7 | + | + | + |
| 2 | 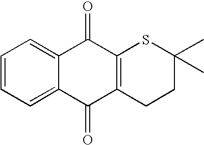 | 3,4-dihydro-2,2-dimethyl- 2H-naphtho[2,3-b]thiopyran- 5,10-dione | — | — | | | |
| 3 | 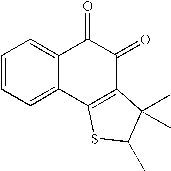 | 2,3,3-trimethyl-2H-3H- naphtho[1,2-b]thiophene- 4,5-dione | 2.0 | — | — | — | — |
| 5 | 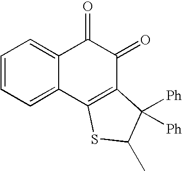 | 2-methyl-3,3-diphenyl-2H- 3H-naphtho[1,2- b]thiophene-4,5-dione | 7.8 | 5.9 | | | |
| 6 | 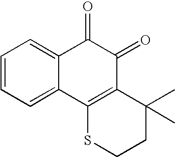 | 3,4-dihydro-4,4-dimethyl- 2H-naphtho[1,2-b]thiopyran 5,6-dione | 6.0 | 6.3 | + | + | + |
| 7 | 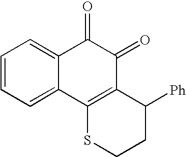 | 3,4-dihydro-4-phenyl-2H- naphtho[1,2-b]thiopyran-5,6 dione | 8.8 | 7 | | | |
| 9 | 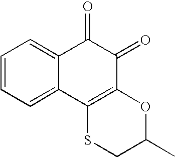 | 2-methyl-11,12-dehydro- naphtho[1,2-b]thioxane- 9,10-dione | 4.9 | 6.4 | — | — | — |
| 10 | 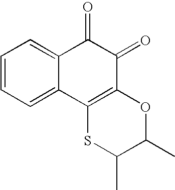 | 2,3-dimethyl-11,12-dehydro naphtho[1,2-b]thioxane- 9,10-dione | 6.1 | 8 | — | — | — |
| 12 | 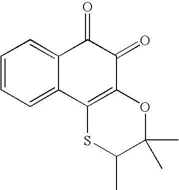 | 2,2,3-trimethyl-11,12- dehydro-naphtho[1,2- b]thioxane-9,10-dione | 13.1 | 14.1 | | | |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A compound of formula I: 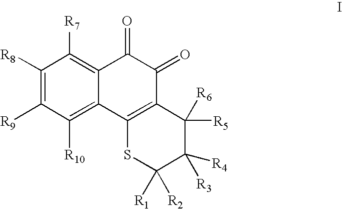
or pharmaceutically acceptable salts thereof, or a regioisomeric mixture thereof, wherein
R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH2)ₙ-amino, —(CH₂)ₙ-aryl, —(CH₂)ₙ-heterocycle, and —(CH₂)ₙ-phenyl; or one of R1 or R2 and one of R3 or R4; or one of R3 or R4 and one of R5 or R6 form a fused ring, wherein the ring has 4-8 ring members;
R7-R10 are each, independently, hydrogen, hydroxyl, halogen, substituted or unsubstituted alkyl, substituted or unsubstituted alkoxy, nitro, cyano or amide; and
n is an integer from 0 to 10.

2. The compound of claim 1, wherein R1 and R2 are alkyl, R3-R6 are, independently, H, OH, halogen, alkyl, alkoxy, substituted or unsubstituted acyl, substituted alkenyl or substituted alkyl carbonyl, and R7-R10 are hydrogen.

3. The compound of claim 1, wherein R1 and R2 are each methyl and R3-R10 are each hydrogen.

4. The compound of claim 1, wherein R1-R4 are each hydrogen, R5 and R6 are each methyl and R7-R10 are each hydrogen.

5. A pharmaceutical composition comprising a therapeutically effective amount of a compound of claim 1 in combination with a pharmaceutically acceptable carrier.

6. A method of treating or preventing cell proliferative disorders comprising administering to a mammal in need thereof a therapeutically effective amount of the pharmaceutical composition of claim 5.

7. The method of claim 6, wherein administration induces sustained elevation of E2F levels in abnormally proliferating cells without affecting E2F levels in normal cells.

8. A method of treating cancer or precancerous conditions or preventing cancer comprising administering to a mammal in need thereof a therapeutically effective amount of a pharmaceutical composition of claim 5.

9. The method of claim 8, wherein administration induces sustained elevation of E2F levels in cancer cells without affecting E2F levels in normal cells.

10. A method of treating or preventing psoriasis comprising administering to a mammal in need thereof a therapeutically effective amount of a pharmaceutical composition of claim 5.

11. A compound of formula II: 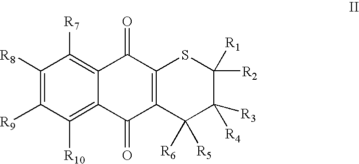
or pharmaceutically acceptable salts thereof, or a regioisomeric mixture thereof, wherein
R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R7-R10 are each, independently, hydrogen, hydroxyl, halogen, substituted or unsubstituted alkyl, substituted or unsubstituted alkoxy, nitro, cyano or amide; and
n is an integer from 0 to 10,
wherein when R1 and R2 are both methyl, R3 and R4 are both H, and one of R5 and R6 is OH and the other H, R7 is not methyl or methoxy and R10 is not methyl.

12. The compound of claim 11, wherein R1 and R2 are alkyl, R3-R6 are independently H, OH, halogen, alkyl, alkoxy, substituted and unsubstituted acyl, substituted alkenyl or substituted alkyl carbonyl, and R7-R10 are hydrogen.

13. The compound of claim 11, wherein R1 and R2 are each methyl and R3-R10 are each hydrogen.

14. A pharmaceutical composition comprising a therapeutically effective amount of a compound of claim 11 in combination with a pharmaceutically acceptable carrier.

15. A method of treating or preventing cell proliferative disorders comprising administering to a mammal in need thereof a therapeutically effective amount of the pharmaceutical composition of claim 14.

16. The method of claim 15, wherein administration induces sustained elevation of E2F levels in abnormally proliferating cells without affecting E2F levels in normal cells.

17. A method of treating cancer or precancerous conditions or preventing cancer comprising administering to a mammal in need thereof a therapeutically effective amount of a pharmaceutical composition of claim 14.

18. The method of claim 17, wherein administration induces sustained elevation of E2F levels in cancer cells without affecting E2F levels in normal cells.

19. A method of treating or preventing psoriasis comprising administering to a mammal in need thereof a therapeutically effective amount of a pharmaceutical composition of claim 14.

20. A compound of formula III: 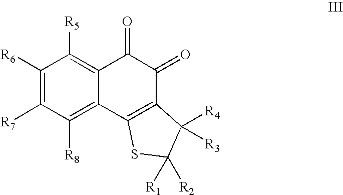
or pharmaceutically acceptable salts thereof, or a regioisomeric mixture thereof, wherein
R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R5-R8 are each, independently, hydrogen, hydroxyl, halogen, substituted or unsubstituted alkyl, substituted or unsubstituted alkoxy, nitro, cyano or amide; and
n is an integer from 0 to 10.

21. The compound of claim 20, wherein one of R1 and R2 is H and the other is alkyl, R3 and R4 are independently H, OH, halogen, alkyl, alkoxy, substituted or unsubstituted alkenyl or substituted or unsubstituted alkyl carbonyl, and R5-R8 are each hydrogen.

22. The compound of claim 20, wherein one of R1 and R2 is H and the other is alkyl, R3 and R4 are each methyl, and R5-R8 are each hydrogen.

23. A pharmaceutical composition comprising a therapeutically effective amount of a compound of claim 20 in combination with a pharmaceutically acceptable carrier.

24. A method of treating or preventing cell proliferative disorders comprising administering to a mammal in need thereof a therapeutically effective amount of the pharmaceutical composition of claim 23.

25. The method of claim 24, wherein administration induces sustained elevation of E2F levels in abnormally proliferating cells without affecting E2F levels in normal cells.

26. A method of treating cancer or precancerous conditions or preventing cancer comprising administering to a mammal in need thereof a therapeutically effective amount of a pharmaceutical composition of claim 23.

27. The method of claim 26, wherein administration induces sustained elevation of E2F levels in cancer cells without affecting E2F levels in normal cells.

28. A method of treating or preventing psoriasis comprising administering to a mammal in need thereof a therapeutically effective amount of a pharmaceutical composition of claim 23.

29. A compound of Formula IV: 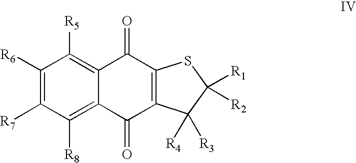
or pharmaceutically acceptable salts thereof, or a regioisomeric mixture thereof, wherein
R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R5-R8 are each, independently, hydrogen, hydroxyl, halogen, substituted or unsubstituted alkyl, substituted or unsubstituted alkoxy, nitro, cyano or amide; and
n is an integer from 0 to 10,
wherein:
when R1 and R2 are both methyl, one of R3 and R4 is not methyl and the other H, and each of R5, R6, R7 and R8 is not H;
when R1 and R2 are both methyl, at least one of R3, R4, R5, R6, R7 and R8 is not H;
when R1 and R2 are both phenyl, at least one of R3, R4, R5, R6, R7 and R8 is not H;
when one of R1 and R2 is phenyl and the other is H, at least one of R3, R4, R5, R6, R7 and R8 is not H;
when one of R1 and R2 is phenyl and the other is methyl, at least one of R3, R4, R5, R6, R7 and R8 is not H;
when one of R1 and R2 is methyl and the other is H and R5, R6, R7 and R8 are H, at least one of R3 and R4 is not OH and the other H;
when one of R3 and R4 are carboethoxy the other is H; and
R1, R2, R3, R4, R5, R6, R7 and R8 are not each H.

30. The compound of claim 29 wherein, both R1 and R2 are substituted or unsubstituted alkyl, R3 and R4 are independently H, OH, halogen, alkyl, alkoxy, substituted or unsubstituted alkenyl or substituted or unsubstituted alkyl carbonyl, and R5-R8 are each hydrogen.

31. The compound of claim 29 wherein, one of R1 and R2 is H and the other is methyl, R3 and R4 are each methyl and R5-R8 are each hydrogen.

32. A pharmaceutical composition comprising a therapeutically effective amount of a compound of claim 29 in combination with a pharmaceutically acceptable carrier.

33. A method of treating or preventing cell proliferative disorders comprising administering to a mammal in need thereof a therapeutically effective amount of the pharmaceutical composition of claim 32.

34. The method of claim 33, wherein administration induces sustained elevation of E2F levels in abnormally proliferating cells without affecting E2F levels in normal cells.

35. A method of treating cancer or precancerous conditions or preventing cancer comprising administering to a mammal in need thereof a therapeutically effective amount of a pharmaceutical composition of claim 32.

36. The method of claim 35, wherein administration induces sustained elevation of E2F levels in cancer cells without affecting E2F levels in normal cells.

37. A method of treating or preventing psoriasis comprising administering to a mammal in need thereof a therapeutically effective amount of a pharmaceutical composition of claim 32.

38. A compound of formula V: 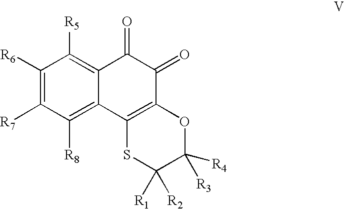
or pharmaceutically acceptable salts thereof, or a regioisomeric mixture thereof, wherein
R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R1 or R2 and one of R3 or R4 form a fused ring, wherein the ring has 4-8 ring members;
R5-R8 are each, independently, hydrogen, hydroxyl, halogen, substituted or unsubstituted alkyl, substituted or unsubstituted alkoxy, nitro, cyano or amide; and
n is an integer from 1 to 10,
wherein R1, R2, R3, R4, R5, R6, R7 and R8 are not each H.

39. The compound of claim 40, wherein R1 and R2 are alkyl, R3-R4 are, independently, H, OH, halogen, alkyl, alkoxy, substituted or unsubstituted acyl, substituted alkenyl or substituted alkyl carbonyl, and R7-R10 are each hydrogen.

40. The compound of claim 40, wherein R1 and R2 are each hydrogen, one of R3 and R4 is methyl and the other is hydrogen and R5-R8 are each hydrogen.

41. The compound of claim 40, wherein one of R1 and R2 is methyl and the other is hydrogen, one of R3 and R4 is methyl and the other is hydrogen and R5-R8 are each hydrogen.

42. The compound of claim 40, wherein one of R1 and R2 is methyl and the other is hydrogen, one of R3 and R4 is hydroxymethyl and the other is hydrogen and R5-R8 are each hydrogen.

43. The compound of claim 40, wherein one of R1 and R2 is methyl and the other is hydrogen, R3 and R4 are each methyl and R5-R8 are each hydrogen.

44. A pharmaceutical composition comprising a therapeutically effective amount of a compound of claim 40 in combination with a pharmaceutically acceptable carrier.

45. A method of treating or preventing cell proliferative disorders comprising administering to a mammal in need thereof a therapeutically effective amount of the pharmaceutical composition of claim 44.

46. The method of claim 45, wherein administration induces sustained elevation of E2F levels in abnormally proliferating cells without affecting E2F levels in normal cells.

47. A method of treating cancer or precancerous conditions or preventing cancer comprising administering to a mammal in need thereof a therapeutically effective amount of a pharmaceutical composition of claim 44.

48. The method of claim 47, wherein administration induces sustained elevation of E2F levels in cancer cells without affecting E2F levels in normal cells.

49. A method of treating or preventing psoriasis comprising administering to a mammal in need thereof a therapeutically effective amount of a pharmaceutical composition of claim 44.

50. A method for the synthesis of a compound of Formula I comprising: reacting a compound having the Formula A: 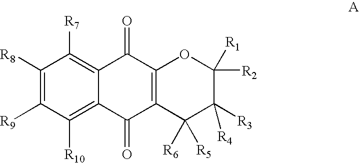
wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R7-R10 are each, independently, hydrogen, hydroxyl, halogen, substituted or unsubstituted alkyl, substituted or unsubstituted alkoxy, nitro, cyano or amide; and
n is an integer from 0 to 10,
with sodium hydrosulfide to exchange oxygen for sulfur and forming the di-thione of the compound of Formula A;
and,
reacting said di-thione of the compound of Formula A with a strong acid to form a compound of Formula I.

51. The method of claim 50, wherein said strong acid is concentrated sulfuric acid.

52. A method for the synthesis of a compound of Formula II comprising: reacting a compound having the Formula B: 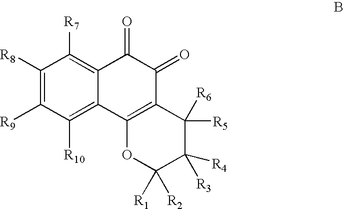
wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R7-R10 are each, independently, hydrogen, hydroxyl, halogen, substituted or unsubstituted alkyl, substituted or unsubstituted alkoxy, nitro, cyano or amide; and
n is an integer from 0 to 10,
wherein when R1 and R2 are both methyl, R3 and R4 are both H, and one of R5 and R6 is OH and the other H, R7 is not methyl or methoxy and R10 is not methyl,
with sodium disulfide to exchange oxygen for sulfur and forming the di-thione of the compound of Formula B;
and,
reacting said di-thione of the compound of Formula B with a strong acid to form a compound of Formula II.

53. The method of claim 52, wherein said strong acid is concentrated sulfuric acid.

54. A method for synthesizing a compound of formula III, comprising reacting a compound having the Formula C: 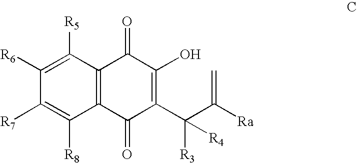
wherein Ra is selected from R1 and R2;
R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R5-R8 are each, independently, hydrogen, hydroxyl, halogen, substituted or unsubstituted alkyl, substituted or unsubstituted alkoxy, nitro, cyano or amide; n is an integer from 0 to 10
with sodium hydrosulfide to form the di-thione of the compound of Formula C,
and,
treating said di-thione of compound of Formula C with a strong acid to form a compound of Formula III.

55. The method of claim 54, wherein said strong acid is concentrated sulfuric acid.

56. A method for synthesizing a compound of formula IV, reacting a compound having the Formula D: 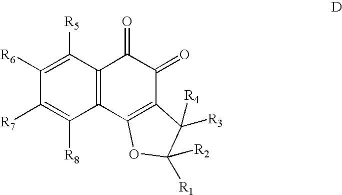
R1-R4 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl;
R5-R8 are each, independently, hydrogen, hydroxyl, halogen, substituted or unsubstituted alkyl, substituted or unsubstituted alkoxy, nitro, cyano or amide; and
n is an integer from 0 to 10,
wherein:
when R1 and R2 are both methyl, one of R3 and R4 is not methyl and the other H, and each of R5, R6, R7 and R8 is not H;
when R1 and R2 are both methyl, at least one of R3, R4, R5, R6, R7 and R8 is not H;
when R1 and R2 are both phenyl, at least one of R3, R4, R5, R6, R7 and R8 is not H;
when one of R1 and R2 is phenyl and the other is H, at least one of R3, R4, R5, R6, R7 and R8is not H;
when one of R1 and R2 is phenyl and the other is methyl, at least one of R3, R4, R5, R6, R7 and R8 is not H;
when one of R1 and R2 is methyl and the other is H and R5, R6, R7 and R8 are H, at least one of R3 and R4 is not OH and the other H;
when one of R3 and R4 are carboethoxy the other is H; and
R1, R2, R3, R4, R5, R6, R7 and R8 are not each H,
with sodium hydrosulfide to form the di-thione of the compound of Formula D,
and,
treating said di-thione of the compound of Formula D with a strong acid to form a compound of Formula IV.

57. The method of claim 56, wherein said strong acid is concentrated sulfuric acid.

58. A method for the synthesis of a compound of Formula I comprising:
reacting a compound having the Formula E: 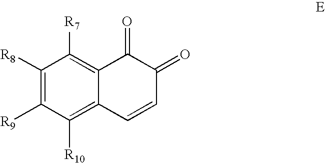
wherein R7-R10 are each, independently, hydrogen, hydroxyl, halogen, substituted or unsubstituted alkyl, substituted or unsubstituted alkoxy, nitro, cyano or amide,
with a branched allylthiol having the formula: 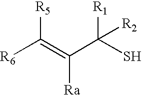
wherein Ra is selected from R3 and R4;
R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; and, and n is an integer from 0 to 10,
in the presence of a weak base to form the sulfide intermediate having the formula: 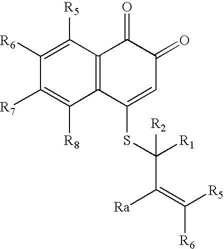
and,
treating said sulfide intermediate with a strong acid to form the a compound of Formula I.

59. The method of claim 58, wherein the weak base is triethylamine.

60. The method of claim 58, wherein said strong acid is concentrated sulfuric acid.

61. A method for the synthesis of a compound of Formula III comprising:
reacting a compound having the Formula F: 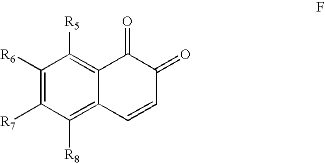
wherein R5-R8 are each, independently, hydrogen, hydroxyl, halogen, substituted or unsubstituted alkyl, substituted or unsubstituted alkoxy, nitro, cyano or amide,
with 2-hydroxyalkylthiol having the formula: 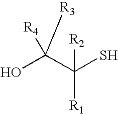
wherein R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxycarbonyl, —(CH₂)ₙ-amino, —(CH₂)ₙ-aryl, —(CH₂)ₙ-heterocycle, —(CH₂)ₙ-phenyl, aryl, heterocycle, and phenyl; and n is an integer from 1 to 10
in the presence of a weak base to form a sulfide intermediate having the formula: 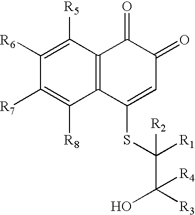
and,
treating said sulfide intermediate with a strong acid to form the a compound of Formula III.

62. The method of claim 61, wherein the weak base is triethylamine.

63. The method of claim 61, wherein said strong acid is concentrated sulfuric acid.

64. A method for synthesizing a compound of Formula V, comprising: reacting a compound having the Formula G: 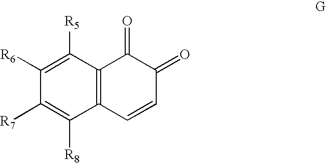
wherein R5-R8 are each, independently, hydrogen, hydroxyl, halogen, substituted or unsubstituted alkyl, substituted or unsubstituted alkoxy, nitro, cyano or amide,
with a 2-hydroxyalcohol having the formula: 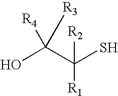
wherein R1-R4 are each, independently, selected from the group consisting of H, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; n is an integer from 1 to 10,
to form a sulfide having the formula: 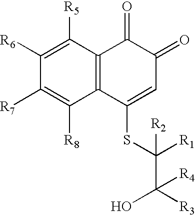
and,
treating said sulfide with strong acid and exposing the reaction to air to form a compound of Formula V.

65. The method of claim 64, wherein said strong acid is concentrated sulfuric acid or trifluoroacetic acid.
